(19) United States
(12) Patent Application Publication
(10) Pub. No.: US 2023/0272330 A1
(43) Pub. Date: Aug. 31, 2023

(54) EVALUATION SYSTEM AND EVALUATION METHOD

(71) Applicant: Hitachi, Ltd., Tokyo (JP)

(72) Inventors: Kakuro HIRAI, Tokyo (JP); Hikaru SAITO, Tokyo (JP); Midori KATO, Tokyo (JP)

(73) Assignee: Hitachi, Ltd., Tokyo (JP)

(21) Appl. No.: 18/081,147

(22) Filed: Dec. 14, 2022

(30) Foreign Application Priority Data
Feb. 25, 2022 (JP) 2022-028184

(51) Int. Cl. C12M 1/34; C12M 1/36; G01N 33/50; G16C 20/70

(52) U.S. Cl. CPC C12M 41/46; C12M 41/48; G01N 33/5005; G16C 20/70

(57) ABSTRACT

Provided are a novel evaluation system and an evaluation method for evaluating a state of cells. The evaluation system includes: a Raman spectroscopic device configured to perform Raman spectroscopic analysis on extracellular vesicles contained in a culture supernatant of the cells; and an analysis device configured to evaluate the state of the cells based on a Raman spectrum obtained by the Raman spectroscopic analysis.

### BACKGROUND OF THE INVENTION

### 1. Field of the Invention

The present invention relates to an evaluation system and an evaluation method.

### 2. Description of the Related Art

Cell culture is generally performed by procedures of a technician in a clean room called a cell processing center (CPC). Since there is a risk of biological contamination in procedure culture in which a culture container is frequently opened and closed, development of a closed automatic culture device that guarantees sterility of a culture environment is promoted.

Cells are affected by a passage number, a cell line, and a slight difference in a culture step which is not completely controlled, and a growth state changes. Therefore, by monitoring a cell state and sequentially optimizing a culture condition, there is a possibility that a better proliferation rate and better differentiation induction efficiency can be achieved.

On the other hand, general evaluation of the cell state is performed by collecting cells and measuring an expression level of a specific marker by flow cytometry, quantitative RT-PCR, or the like. Since the technique involves collection of cells, it is difficult to apply the technique to monitoring.

As a method for evaluating the cell state while the cells are alive, a method of measuring a component in a culture supernatant that can be recovered from a cell culture system is proposed. For example, WO2015166845A1 (Patent Literature 1) discloses that “a stem cell whose differentiation state is unknown or a cell subjected to differentiation induction from a stem cell is set as a test cell, and the differentiation state of the test cell is evaluated based on abundance of a predetermined substance in a culture supernatant of the test cell”. Also, US2021-0301248 (Patent Literature 2) discloses that “a content of exosome markers in a culture supernatant changes in three patterns according to a differentiation induction process of cells”.

### SUMMARY OF THE INVENTION

An object of the invention is to provide a novel evaluation system and evaluation method for evaluating a state of cells.

An embodiment according to the invention is an evaluation system for evaluating a state of cells. The evaluation system includes: a Raman spectroscopic device configured to perform Raman spectroscopic analysis on extracellular vesicles contained in a culture supernatant of the cells; and an analysis device configured to evaluate the state of the cells based on a Raman spectrum obtained by the Raman spectroscopic analysis. The cells may be pluripotent stem cells, dopamine neural progenitor cells, Ectoderm cells, or Mesoderm cells. The state of the cells may be a differentiation stage of the cells. The extracellular vesicles may be exosomes. In the Raman spectrum, the state of the cells may be evaluated based on a measurement result in at least one range selected from the group consisting of 713±10, 830±10, 858±10, 885±10, 895±10, 919±10, 942±10, 997±10, 1040±10, 1065±10, 1107±10, 1133±10, 1175±10, 1299±10, 1372±10, 1420±10, 1443±10, 1739±10, 2663±10, 2730±10, 2850±10, 2887±10, 2936±10, and 2964±10 cm⁻¹. The state of the cells may be evaluated using an evaluation model generated by training data including a set of the Raman spectrum and data of the state of the cells corresponding to the Raman spectrum. A display device may be provided.

Another embodiment according to the invention is an automatic culture system including: any one of the evaluation systems described above and an automatic culture device configured to culture the cells.

A further embodiment according to the invention is an evaluation method for evaluating a state of cells. The method includes: performing Raman spectroscopic analysis on extracellular vesicles isolated from a culture supernatant of the cells; and evaluating the state of the cells based on a Raman spectrum obtained by the Raman spectroscopic analysis. The state of the cells may be evaluated using an evaluation model generated by training data including a set of the Raman spectrum and data of the state of the cells corresponding to the Raman spectrum. The cells may be pluripotent stem cells, dopamine neural progenitor cells, Ectoderm cells, or Mesoderm cells. The state of the cells may be a differentiation stage of the cells. The extracellular vesicles may be exosomes. In the Raman spectrum, the state of the cells may be evaluated based on a measurement result in at least one range selected from the group consisting of 713±10, 830±10, 858±10, 885±10, 895±10, 919±10, 942±10, 997±10, 1040±10, 1065±10, 1107±10, 1133±10, 1175±10, 1299±10, 1372±10, 1420±10, 1443±10, 1739±10, 2663±10, 2730±10, 2850±10, 2887±10, 2936±10, and 2964±10 cm⁻¹.

According to the invention, it is possible to provide a novel evaluation system and an evaluation method for evaluating a state of cells. Problems, configurations, and effects other than those described above will be described in the following embodiments.

### BRIEF DESCRIPTION OF THE DRAWINGS

FIG. 1 is a schematic diagram showing a configuration of an evaluation system according to an embodiment of the invention;

FIG. 2 is a schematic diagram showing a user interface displayed on a display in the evaluation system according to the embodiment of the invention;

FIG. 3 is a control flowchart by an analysis device in the evaluation system according to the embodiment of the invention;

FIG. 4 is a schematic diagram of an automatic culture system including an automatic culture device according to the embodiment of the invention;

FIG. 5 is a representative Raman spectrum of an extracellular vesicle fraction isolated from a culture supernatant of iPS cells according to one example of the invention;

FIG. 6 is a scatter diagram showing a change in a Raman spectrum of extracellular vesicles isolated from a culture supernatant in a differentiation culture step from the iPS cells to dopamine neural progenitor cells according to one example of the invention;

FIG. 7 is a scatter diagram showing a change in a Raman spectrum of extracellular vesicles isolated from a culture supernatant in a differentiation culture step from the iPS cells to Ectoderm cells and Mesoderm cells according to one example of the invention; and

FIG. 8 is a scatter diagram showing a change in a Raman spectrum derived from extracellular vesicles isolated from a culture supernatant under a culture condition of the iPS cells according to one example of the invention.

### DESCRIPTION OF THE PREFERRED EMBODIMENTS

An embodiment of the invention will be described below with reference to the drawings. However, the embodiment is merely an example for implementing the invention, and does not limit the technical scope of the invention.

### Evaluation System

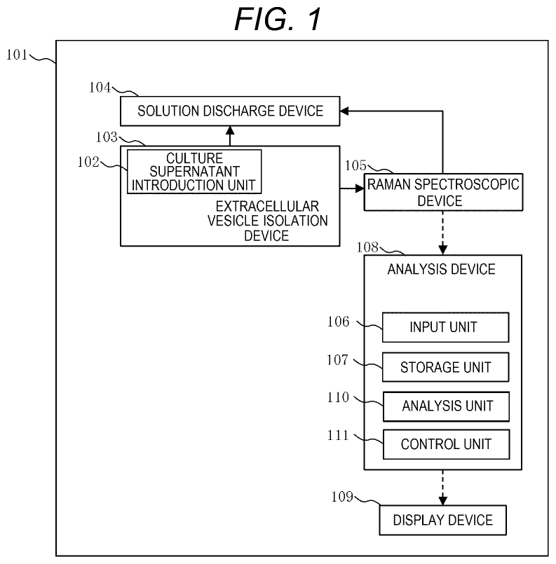

FIG. 1 is a schematic diagram of an evaluation system 101 according to the invention. The evaluation system 101 for evaluating a state of cells includes a Raman spectroscopic device 105 for performing Raman spectroscopic analysis on extracellular vesicles contained in a culture supernatant of the cells, and an analysis device 108 for evaluating the state of the cells based on a Raman spectrum obtained by the Raman spectroscopic analysis.

The analysis device 108 includes a CPU and the like. The CPU includes an input unit 106 for inputting a Raman spectrum, which is obtained by the Raman spectroscopic analysis in the Raman spectroscopic device 105 for inputting the Raman spectrum, and data of the state of the cells from which the Raman spectrum is obtained, a storage unit 107 including a database in which the Raman spectrum and the data of the state of the cells from which the Raman spectrum is obtained are associated with each other, an evaluation program for evaluating the state of the cells based on the Raman spectrum, and the like, an analysis unit 110 for evaluating the state of the cells based on the Raman spectrum obtained by the Raman spectroscopic analysis, and a control unit 111 that controls an operation of each device of the evaluation system 101.

The evaluation system 101 may further include devices such as an extracellular vesicle isolation device 103 for isolating extracellular vesicles from the culture supernatant, a solution discharge device 104 for discharging an unnecessary solution, and a display device 109 such as a display for displaying an analysis result.

### Automatic Culture System

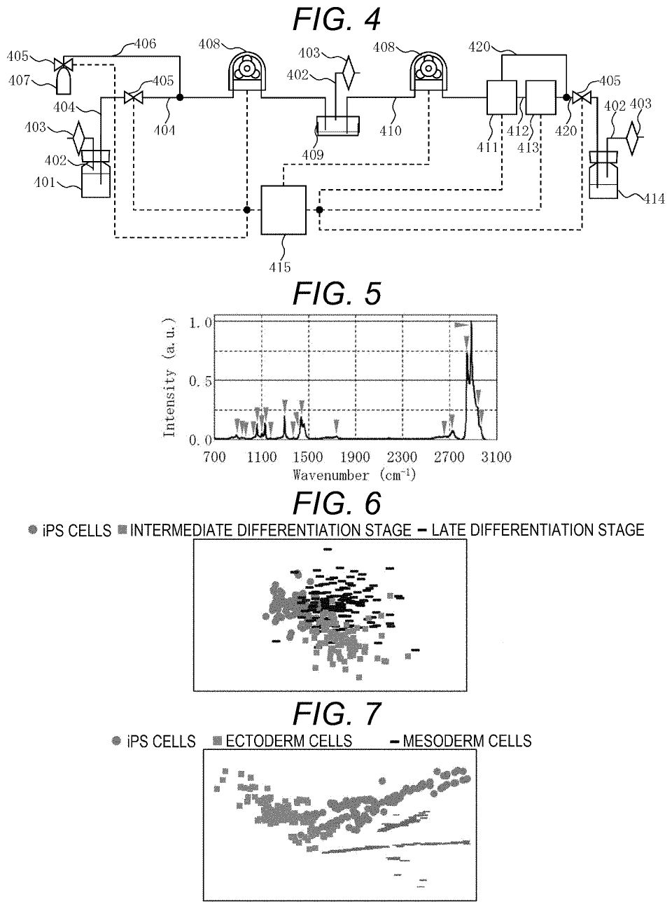

FIG. 4 is a schematic diagram of an automatic culture system including automatic culture devices (including 401 to 409) and evaluation systems (including 410 to 420).

A cell culture solution is supplied from the culture solution storage container 401 to the culture container 409 via the solution supply flow path 404. Pressure in a culture environment is maintained by the atmospheric pressure adjustment flow path 402 and the outside air filter 403. Various gas concentrations in the culture environment are adjusted by the gas cylinder 407 and the gas supply flow path 406. Movements of a culture solution and gas are adjusted by the valve 405 and the pump 408.

The culture supernatant is supplied from the culture container 409 to the extracellular vesicle isolation device 411 via the solution discard flow path 410. The extracellular vesicle isolation device 411 receives the culture supernatant in a culture supernatant introduction unit 102, and isolates the extracellular vesicles from the culture supernatant. An isolation method is not particularly limited, and a known method can be used. The extracellular vesicles are supplied to the Raman spectroscopic device 413 via the sample flow path 412. Waste solutions generated in the extracellular vesicle isolation device 411 and the Raman spectroscopic device 413 are discarded into the waste solution storage container 414 via the solution discard flow path 420. As an analysis device, the terminal 415 that performs device control, data analysis, and data storage controls various valves, pumps, and devices, and stores and analyzes the Raman spectrum obtained by the Raman spectroscopic device 413. Based on a monitoring result obtained by the terminal 415, cell culture is continued, stopped, and the condition is changed.

Determination may be performed by an operator, or may be performed by the analysis device in accordance with a preset standard.

### Control Flow by Analysis Device

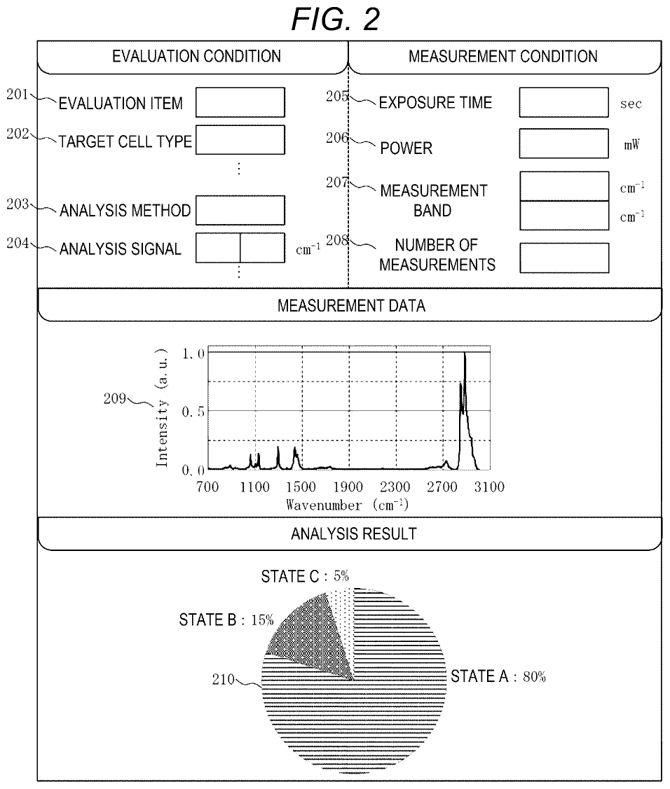

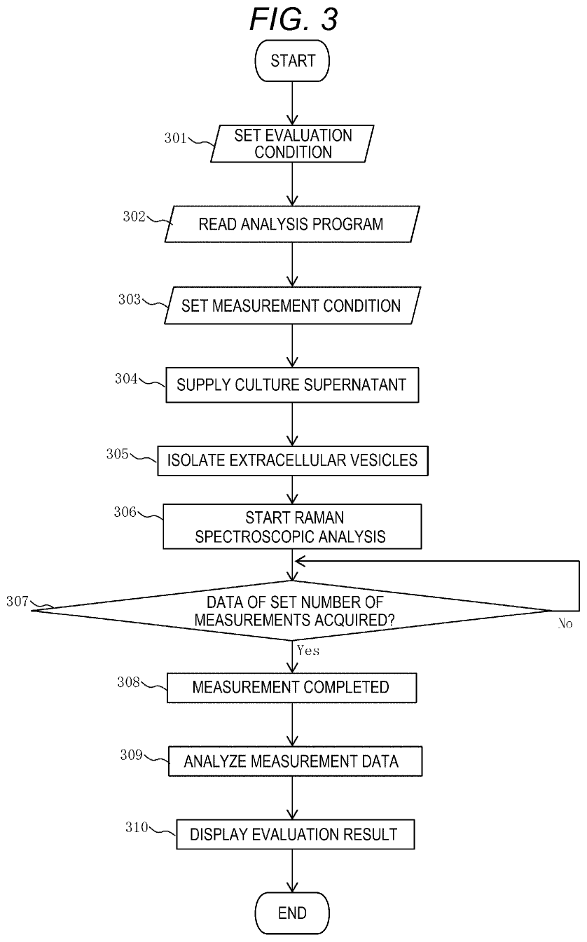

FIG. 2 is a schematic diagram of an example of a user interface displayed on the display device 109. FIG. 3 is a control flowchart of the control unit 111 of the evaluation system 101.

First, the operator sets an evaluation condition by inputting, for example, an item 201 and a cell type 202 of a cell state to be evaluated (301). It is preferable that the control unit 111 reads an analysis program stored in the storage unit 107 according to the set evaluation condition (302) and displays an analysis method 203 and a Raman signal 204 to be analyzed according to the analysis program. The operator may manually input the analysis method 203 and the Raman signal 204 to be analyzed. Next, the operator sets a measurement condition by inputting, for example, a laser exposure time 205, a laser power 206, a Raman signal band 207 to be measured, and the number of measurement data 208 when one spectrum is acquired (303).

The control unit 111 supplies the culture supernatant from the culture container 409 to the extracellular vesicle isolation device 411 (304). Subsequently, the control unit 111 performs control as follows.

The extracellular vesicle isolation device 411 that obtains the culture supernatant isolates the extracellular vesicles from the culture supernatant (305). The extracellular vesicle isolation device 411 sends the isolated extracellular vesicles to the Raman spectroscopic device 105. The Raman spectroscopic device 105 starts the Raman spectroscopic analysis of the extracellular vesicles (306). When the Raman spectroscopic device 105 acquires the data of the set number of measurements (307), the measurement is ended (308). When the Raman spectroscopic device 105 sends measurement data to the analysis device 108, the analysis device 108 receives the measurement data at the input unit 106 and performs the analysis at the analysis unit 110. The measurement data may be temporarily stored in the storage unit 107 before the analysis. The analysis device 108 sends a result obtained by the analysis unit 110 to the display device 109 together with the measurement data. The display device 109 displays measurement data 209 and a result 210 to the operator. In the display device 109, the analysis result may be displayed as percentages of the cell state based on a classification of each Raman spectrum measured.

### Evaluation Method of Cell State

In the invention, the cell state is evaluated based on the Raman spectrum of the extracellular vesicles in the culture supernatant. The evaluation method is not particularly limited, and the method described below is an example. In the invention, the cell state can be exemplified by a cell differentiation stage, cell life and death, a cell division stage, activation of specific signal transduction, gene introduction, and the like. For example, when the state of the cells represents the cell differentiation stage, a method of specifying the cell differentiation stage is not particularly limited. Examples thereof include specifying the differentiation stage by a predetermined number of days after the start of differentiation, specifying the differentiation stage by expression of a predetermined differentiation marker, and specifying the differentiation stage by taking a predetermined cell form. A differentiation direction of the cells is not particularly limited, and may be differentiation into specific germ layers such as an inner germ layer, Mesoderm cells, and Ectoderm cells, or may be differentiation into specific cell types such as nerve cells, fibroblasts, epithelial cells, and blood cells. The cell type to be differentiated is not particularly limited. Examples thereof include undifferentiated cells such as pluripotent stem cells such as iPS cells and ES cells, stem cells such as neural stem cells and mesenchymal stem cells, and precursor cells such as nerve precursor cells and bone marrow precursor cells.

Extracellular vesicles are isolated from cells whose cell state is specified in advance, and a plurality of Raman spectrum data thereof are used as training data to construct a cell state evaluation program using numerical analysis such as multivariate analysis, machine learning, and deep learning. At this time, as specific training data, it is conceivable to use the whole or a part of a waveform of the Raman spectrum, or a characteristic waveform portion. Raman spectroscopy is not limited. Examples thereof include spontaneous Raman scattering, surface-enhanced Raman scattering, and nonlinear Raman scattering.

When a Raman spectrum of extracellular vesicles isolated from a culture supernatant of iPS cells and cells differentiated therefrom is used, at least one Raman spectrum in a wavelength region selected from the group consisting of 713±10, 830±10, 858±10, 885±10, 895±10, 919±10, 942±10, 997±10, 1040±10, 1065±10, 1107±10, 1133±10, 1175±10, 1299±10, 1372±10, 1420±10, 1443±10, 1739±10, 2663±10, 2730±10, 2850±10, 2887±10, 2936±10, and 2964±10 cm⁻¹ may be used.

Then, Raman spectrum data is acquired for cells for which the cell state is to be evaluated, Raman spectrum spectroscopic analysis is performed in the same manner as the training data, and the Raman spectrum data is substituted into the cell state evaluation program. Therefore, the cell state of the cells can be evaluated.

### EXAMPLES

### Example 1

In the present example, it is shown that a pattern of a Raman spectrum for the extracellular vesicles changes in a differentiation induction process of stem cells. Specifically, differentiation induction culture into dopamine neural progenitor cells was performed for 12 days using an iPS cell line 201B7, and a change in the Raman spectrum of extracellular vesicles was evaluated. A differentiation induction condition into dopamine neural progenitor cells was as follows (Stem Cell Reports, vol. 2 (2014), pp. 337-350).

First, feeder-free iPS cells cultured in StemFit media (Ajinomoto Co., Ltd.) were treated with TrypLE select for 10 minutes, and then separated into single cells. The iPS cells were then seeded onto an LM511-E8-coated 6-well plate at a density of 4×10⁵ cells and cultured for 4 days to reach a confluent state. Therefore, a medium was changed to a differentiation medium containing GMEM to which 8% KSR, 0.1 mM MEM non-essential amino acids (all manufactured by Invitrogen Co., Ltd.), sodium pyruvate (Sigma-Aldrich Co., Ltd.), and 0.1 mM 2-mercaptoethanol were added, and differentiation induction was started. In order to induce nerve differentiation more efficiently, LDN 193189 (STEMGENT) and A83-01 (Wako) were added to the medium (J. Neurosci. Res., vol. 89 (2011), pp. 117-126).

Such differentiation induction culture from the iPS cells to the dopamine nerve progenitor cells was similarly performed in 3 wells of the 6-well plate, and the culture supernatant of the iPS cells on a start date of the differentiation induction and the culture supernatants on the 8-th day after differentiation induction transition (intermediate differentiation stage) and the 12-th day after the differentiation induction transition (late differentiation stage) were collected. Fraction of the isolated extracellular vesicle was subjected to Raman spectroscopy by using a phosphatidylserine affinity method (Sci Rep., vol. 6 (2016), pp 33935). In the analysis of the Raman spectrum, wavenumbers around a signal shown in the Raman spectrum in FIG. 5 are 881.0, 883.1, 885.1, 887.2, 889.2, 891.3, 893.3, 895.4, 897.4, 899.5, 909.7, 911.7, 913.8, 915.8, 917.8, 919.9, 921.9, 924.0, 926.0, 928.0, 930.1, 932.1, 934.2, 936.2, 938.2, 940.3, 942.3, 944.3, 946.4, 948.4, 950.4, 952.5, 986.9, 989.0, 991.0, 993.0, 995.0, 997.1, 999.1, 1001.1, 1003.1, 1005.1, 1007.2, 1029.3, 1031.4, 1033.4, 1035.4, 1037.4, 1039.4, 1041.4, 1043.4, 1045.4, 1047.5, 1049.5, 1055.5, 1057.5, 1059.5, 1061.5, 1063.5, 1065.5, 1067.5, 1069.5, 1071.5, 1073.5, 1075.5, 1097.5, 1099.5, 1101.5, 1103.5, 1105.5, 1107.5, 1109.5, 1111.5, 1113.5, 1115.5, 1117.5, 1123.5, 1125.5, 1127.5, 1129.5, 1131.4, 1133.4, 1135.4, 1137.4, 1139.4, 1141.4, 1143.4, 1165.2, 1167.2, 1169.2, 1171.1, 1173.1, 1175.1, 1177.1, 1179.1, 1181.0, 1183.0, 1185.0, 1289.1, 1291.1, 1293.0, 1295.0, 1296.9, 1298.9, 1300.8, 1302.8, 1304.7, 1306.7, 1308.6, 1310.6, 1363.0, 1364.9, 1366.8, 1368.8, 1370.7, 1372.6, 1374.6, 1376.5, 1378.4, 1380.4, 1382.3, 1409.3, 1411.2, 1413.1, 1415.0, 1417.0, 1418.9, 1420.8, 1422.7, 1424.6, 1426.6, 1428.5, 1430.4, 1432.3, 1434.2, 1436.2, 1438.1, 1440.0, 1441.9, 1443.8, 1445.7, 1447.7, 1449.6, 1451.5, 1453.4, 1729.9, 1731.8, 1733.6, 1735.5, 1737.3, 1739.2, 1741.0, 1742.9, 1744.7, 1746.6, 1748.4, 1750.3, 2653.3, 2654.9, 2656.5, 2658.2, 2659.8, 2661.5, 2663.1, 2664.7, 2666.4, 2668.0, 2669.7, 2671.3, 2672.9, 2720.2, 2721.9, 2723.5, 2725.1, 2726.7, 2728.4, 2730.0, 2731.6, 2733.2, 2734.9, 2736.5, 2738.1, 2739.7, 2839.6, 2841.2, 2842.8, 2844.4, 2846.0, 2847.6, 2849.2, 2850.8, 2852.4, 2854.0, 2855.6, 2857.2, 2858.8, 2860.4, 2876.3, 2877.9, 2879.5, 2881.1, 2882.7, 2884.3, 2885.9, 2887.4, 2889.0, 2890.6, 2892.2, 2893.8, 2895.4, 2897.0, 2925.5, 2927.1, 2928.7, 2930.2, 2931.8, 2933.4, 2935.0, 2936.6, 2938.1, 2939.7, 2941.3, 2942.9, 2944.4, 2946.0, 2952.3, 2953.9, 2955.5, 2957.1, 2958.6, 2960.2, 2961.8, 2963.3, 2964.9, 2966.5, 2968.1, 2969.6, 2971.2, 2972.8, and 2974.4 cm⁻¹.

Among the cultured 3 wells, measurement data for 2 wells were used as the training data, and measurement data for 1 well was used as test data. Data features were extracted by independent component analysis using the training data, and a classifier was created by logistic regression. In the independent component analysis, dimensions of data were three-dimensionally compressed. The dimensions of the independent component analysis used in the classifier and a hyperparameter of the logistic regression were optimized by a 10-division cross-validation method. As a result of the optimization, the number of dimensions of the independent component analysis used in the classifier was 2. FIG. 6 shows a scatter diagram of an independent component analysis result of all measured spectrum data.

As shown in the graph of FIG. 6, the spectrum data was separated in each of an intermediate stage and a late stage of differentiation induction from iPS cells to dopaminergic neural progenitor cells. For the analysis, scikit-learn (0.23.1), which is a machine learning library of Python, was used.

As a result of evaluating the test data by the created classifier, a classification accuracy F value of the data was 86%.

As described above, a composition of the extracellular vesicles changes depending on the differentiation state of the cells, and the change is reflected in the Raman spectrum of the extracellular vesicles.

### Example 2

In the present example, it is shown that a pattern of the Raman spectrum derived from the extracellular vesicles changes even in a differentiation induction system different from that of Example 1. Specifically, differentiation induction culture into Ectoderm cells and Mesoderm cells was performed using the iPS cell line 201B7, and a change in the Raman spectrum of the extracellular vesicles was evaluated. Differentiation induction into both germ layers was performed using STEM diff Trilineage Differentiation Kit (R).

Differentiation induction culture from the iPS cells to the Ectoderm cells and the Mesoderm cells was performed in 2 wells of the 6-well plate, and the culture supernatant of the iPS cells on a start date of the differentiation induction and the culture supernatant on a final date of the differentiation induction of each germ layer were collected, and extracellular vesicles isolated therefrom were subjected to the Raman spectroscopy. The Raman spectrum was analyzed in the same manner as in Example 1. FIG. 7 is a scatter diagram showing an independent component analysis result of all spectrum data of the iPS cells, the Ectoderm cells, and the Mesoderm cells.

As shown in the graph of FIG. 7, the spectrum data of the iPS cells and both germ layers tended to separate.

One of the cultured 2 wells was set as training data and the other was set as test data, and the classification accuracy evaluated in the same manner as in Example 1 was 87%.

As described above, even in the differentiation induction system different from that of Example 1, the differentiation state of the cells can be evaluated by the Raman spectrum of the extracellular vesicles.

### Example 3

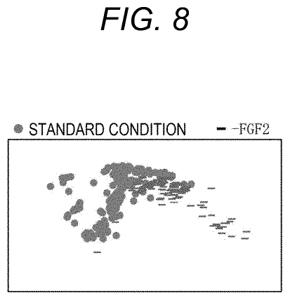

In the present example, it is shown that the Raman spectrum of the extracellular vesicles is changed by deviating from an undifferentiated state of the stem cell. Specifically, in the culture of proliferating the iPS cell line 201B7, in order to deviate from the undifferentiated state in parallel with a standard culture condition, the iPS cells were cultured under a condition (hereinafter, referred to as a deviation condition) in which a basic fibroblast growth factor (FGF2) necessary for maintaining the undifferentiated state of the iPS cells was removed from the culture medium. Both conditions were performed for each of 3 wells of the 6-well plate. Under the deviation condition, the iPS cells were seeded in the same manner as in Example 1, and then the medium was replaced with a medium from which the FGF2 was removed on the 3-th day. Under both culture conditions, the cells and the culture supernatant were collected on the 7-th day from the start of the culture. As a result of evaluating an expression level of an undifferentiated marker Nanog of the cells by quantitative RT-PCR, the expression level was significantly reduced under the deviation condition compared to the standard condition. The Raman spectroscopy and the Raman spectrum analysis were performed in the same manner as in Example 1. FIG. 8 is a scatter diagram of an independent component analysis result of all spectrum data under both culture conditions.

As shown in the graph of FIG. 8, the spectrum data tended to separate for each culture condition. The classification accuracy evaluated in the same manner as in Examples 1 and 2 was 85%.

Therefore, the undifferentiated state of the stem cells can be evaluated by the Raman spectrum derived from the extracellular vesicles.

What is claimed is:

1. An evaluation system for evaluating a state of cells, the evaluation system comprising:
a Raman spectroscopic device configured to perform Raman spectroscopic analysis on extracellular vesicles contained in a culture supernatant of the cells; and
an analysis device configured to evaluate the state of the cells based on a Raman spectrum obtained by the Raman spectroscopic analysis.

2. The evaluation system according to claim 1, wherein
the cells are pluripotent stem cells, dopamine neural progenitor cells, Ectoderm cells, or Mesoderm cells.

3. The evaluation system according to claim 1, wherein
the state of the cells is a differentiation stage of the cells.

4. The evaluation system according to claim 1, wherein
the extracellular vesicles are exosomes.

5. The evaluation system according to claim 1, wherein
in the Raman spectrum, the state of the cells is evaluated based on a measurement result in at least one range selected from the group consisting of 713±10, 830±10, 858±10, 885±10, 895±10, 919±10, 942±10, 997±10, 1040±10, 1065±10, 1107±10, 1133±10, 1175±10, 1299±10, 1372±10, 1420±10, 1443±10, 1739±10, 2663±10, 2730±10, 2850±10, 2887±10, 2936±10, and 2964±10 cm⁻¹.

6. The evaluation system according to claim 1, wherein
the state of the cells is evaluated using an evaluation model generated by training data including a set of the Raman spectrum and data of the state of the cells corresponding to the Raman spectrum.

7. The evaluation system according to claim 1, further comprising:
a display device.

8. An automatic culture system comprising:
the evaluation system according to claim 1; and
an automatic culture device configured to culture the cells.

9. An evaluation method for evaluating a state of cells, the method comprising:
performing Raman spectroscopic analysis on extracellular vesicles isolated from a culture supernatant of the cells; and
evaluating the state of the cells based on a Raman spectrum obtained by the Raman spectroscopic analysis.

10. The evaluation method according to claim 9, wherein
the state of the cells is evaluated using an evaluation model generated by training data including a set of the Raman spectrum and data of the state of the cells corresponding to the Raman spectrum.

11. The evaluation method according to claim 9, wherein
the cells are pluripotent stem cells, dopamine neural progenitor cells, Ectoderm cells, or Mesoderm cells. cells.

12. The evaluation method according to claim 9, wherein
the state of the cells is a differentiation stage of the

13. The evaluation method according to claim 9, wherein
the extracellular vesicles are exosomes.

14. The evaluation method according to claim 9, wherein
in the Raman spectrum, the state of the cells is evaluated based on a measurement result in at least one range selected from the group consisting of 713±10, 830±10, 858±10, 885±10, 895±10, 919±10, 942±10, 997±10, 1040±10, 1065±10, 1107±10, 1133±10, 1175±10, 1299±10, 1372±10, 1420±10, 1443±10, 1739±10, 2663±10, 2730±10, 2850±10, 2887±10, 2936±10, and 2964±10 cm⁻¹.
